(19) United States
(12) Patent Application Publication
(10) Pub. No.: US 2024/0091101 A1
(43) Pub. Date: Mar. 21, 2024

(54) CLOSURE SYSTEM FOR A MEDICAMENT CONTAINER, AND MEDICAMENT CONTAINER HAVING A CLOSURE SYSTEM

(71) Applicant: TRENTA2 S.R.L., Bozen (IT)

(72) Inventor: Dietrich GALLMETZER, Terlan (IT)

(73) Assignee: TRENTA2 S.R.L., Bozen (IT)

(21) Appl. No.: 18/038,443

(22) PCT Filed: Nov. 24, 2021
(86) PCT No.: PCT/EP2021/082866

§ 371 Date: May 24, 2023

(30) Foreign Application Priority Data
Nov. 24, 2020 (EP) 20209611.1

(51) Int. Cl. A61J 1/14; A61J 1/18; B65D 41/58; B65D 51/00

(52) U.S. Cl. CPC A61J 1/1425; A61J 1/1468; A61J 1/18; B65D 41/58; B65D 51/002; A61J 2205/60; B65D 2401/15

(57) ABSTRACT

A closure system for a medicament container designed in the manner of a bottle mouth is intended to seal against unwanted and undesired material loss. The closure system comprises: a press-fit cap with an annular cover which has a central opening and at whose outer circumference latch elements are arranged which can be brought into engagement with an outer bead fitted peripherally on the container opening, and a closure stopper with a closure plate, on the first plate side of which there is integrally formed a thickened portion which completely fills the central opening of the annular cover and can be brought into latching engagement therewith, and on the second plate side of which there is integrally formed a radial sealing element having a cross section which is adapted to the clear width of the container opening and is slightly larger than the clear width of the container opening.

### FIELD OF INVENTION

The invention relates to a closure system for a medicament container, the interior of which can be accessed via a container opening in the form of a bottle mouth. It further relates to a medicament container with such a closure system and to a use of the closure system.

### BACKGROUND

Medications, particularly for treatment in highly specialized or complex therapies, are usually provided in drug or medication containers, also referred to as containers or vials. Such a drug container is usually designed in the manner of a vial and comprises an interior space in which the drug or active ingredient is kept and which is accessible via a container opening designed in the manner of a bottle mouth. From such a container or receptacle, the active ingredient is then transferred via suitable transfer systems for the actual administration to suitable systems such as a syringe or an intravenous line that provides fluid access to the patient's blood system.

Modern medical procedures or therapies in particular may involve the use of drugs or substances that are actually toxic or otherwise harmful or hazardous in themselves. Acute and long-term health risks can thus arise for personnel entrusted with handling such substances, such as pharmacists and nurses, especially if they are repeatedly exposed to drugs or solvents that could escape into the air during preparation, administration of medicines, and other similar treatments. This problem can be particularly severe when cytotoxins, antiviral medications, antibiotics, or radiopharmaceuticals are involved. Health risks potentially arising from exposure to these drugs include increased risk of cancer, genetic alterations, and the like.

Furthermore, also in view of the fact that drugs with an extremely high dose price have recently been approved, it is urgently desirable or even necessary to reliably prevent the unintentional release of even the smallest quantities of such medicines or active ingredients into the environment. For this purpose, so-called closed transfer systems are in common use, in which it is ensured in the manner of an encapsulated design in every phase of the liquid transfer that the active substance or the gases or aerosols released by it cannot escape into the environment.

However, for proper operation of such systems with the desired prevention of unintentional loss of active ingredients, the drug containers must also be suitably designed. For this purpose, the active ingredient or drug containers are usually equipped with suitable closure systems in which a closure stopper closes the container opening. This stopper can then be pierced for removal of the drug, for example by means of a hollow needle, via which the drug can then be sucked out of the container. To fix the sealing stopper, a press-fit cap with an annular cover having a central opening can be provided, which can be attached to the “bottle mouth” with the container opening. The sealing stopper is then fitted centrally in the ring cap.

Even with such closure systems, however, it has been found that it is not possible to guarantee the desired tightness of the system in all cases and thus to prevent as far as possible the undesirable release of drugs or active ingredients.

### BRIEF SUMMARY

It is therefore an object of the present invention to provide a closure system of the type mentioned above, with which the overall tightness of the system can be further improved. Furthermore, a medicament container with such a closure system and a particularly favorable use of such a closure system shall be provided.

With regard to the closure system for a medicament container, the interior of which is accessible via a container opening configured in the manner of a bottle mouth, this object is achieved according to the invention in that the closure system comprises:
a. a press-fit cap with an annular lid having a central opening, on the outer periphery of which a number of latching elements are arranged which can be brought into engagement with an outer bead arranged circumferentially on the container opening, and
b. a closure stopper having a closure plate, on the first plate side of which there is integrally formed a thickened portion which completely fills the central opening of the annular cover and can be brought into engagement therewith, and on the second plate side of which there is integrally formed a radial sealing element having a cross-section which is adapted to the clear width of the container opening and is selected to be slightly larger than the clear width of the container opening with respect to the deformability of the material of the closure stopper.

Advantageous embodiments of the invention are specified in the subclaims.

The invention is based on the consideration that the sealing effect of the sealing stopper should be designed to be particularly comprehensive in order to achieve a particularly high tightness of the overall system and to avoid undesirable losses of active ingredient to a particularly large extent. Surprisingly, several aspects have to be taken into account in the design of the sealing stopper. In conventional systems, the sealing stopper, usually based on a rubber-like or sufficiently elastic material, is designed to be inserted into the container opening and placed on its boundary or mouth edge. By means of an associated fastening element, for example a suitable baffle cap that can be snapped onto the mouth edge, the edge of the closure stopper is then pressed onto the mouth edge of the container opening so that it can develop its sealing effect as a result of the deformability of the material. However, such a system only uses axial force components for the sealing effect, i.e. force components in a direction parallel to the longitudinal or central axis of the “bottle mouth” of the container.

In order to further increase the sealing effect that can be achieved in this way, however, radial force components, i.e. contact forces that press the closure stopper radially against the inside of the container mouth, should also be made available. In order to take this into account, the shape and dimensions of the cross-section of the sealing stopper in the area projecting into the container opening should be such that, taking into account the deformability of its material, a flat pressure or contact pressure effect is produced on the inner wall of the container opening.

The closure stopper can be of multi-component construction, with a first, preferably centrally arranged component being designed as a piercing surface for a needle or other transfer system. Thereby, by piercing this first central component, an access to the interior of the container can be established, through which active ingredient can be removed. Subsequently, the needle can be withdrawn again from the thus formed piercing membrane, whereby the pierced surface closes again, preferably sealingly, due to the elastic properties of the stopper material. In addition to this, a second component of the closure stopper is advantageously provided, which is designed as a sealing element in the radial direction and advantageously surrounds the said first component radially on the outside. Due to the functional separation of the two components, in particular a functionally selected different design of both components, for example with regard to the choice of material and/or the specific material properties, can be provided with regard to the respective desired properties. With such a multi-component, preferably two-component, design of the closure stopper, in combination with the press-fit cap, in particular after pre-assembly of the closure stopper and press-fit cap with each other, this results in a so-called three-component (“3K”) design of the pre-assembled intermediate product.

In an alternative advantageous embodiment, the sealing stopper is of one-piece design, so that the intermediate product formed after pre-assembly of the sealing stopper and the press-fit cap with each other has a so-called two-component (“2K”) design.

Advantageously, the components are particularly adapted to the intended functionality with regard to their choice of material: the press-fit cap should be suitable for the intended snap-on or latching connection on the container opening or a latching edge surrounding it and, in particular, should be of correspondingly rigid design. In contrast, the sealing stopper should be designed to be suitable for the desired slight deformation during the creation of the sealing effect. Accordingly, the press-fit cap is preferably made of a plastic, particularly preferably polypropylene (PP), a polyolefin, cyclo-olefin copolymer (COC), cyclo-olefin polymer (COP), or polycarbonate. In an alternative or additional advantageous embodiment, the sealing stopper is made of rubber or of TPE. Particularly in the case of the above-mentioned multi-component design of the sealing stopper, different material or rubber mixtures can be provided for the respective components, each of which is suitably selected with regard to the intended function.

For particularly safe and reliable handling, the closure system should be specially secured after it has been attached to the medication container. For this purpose, it advantageously includes as a further component a locking ring which can be pushed onto the press-fit cap and which can be fixed in a latching manner to the press-fit cap by means of a snap-on rim formed on the inside and arranged around the end of the press-fit cap. After the press-fit cap has been fitted, in which the latching elements arranged on its outer circumference are brought into engagement, i.e. “snapped on” or engaged, with the outer bead fitted around the container opening when it is pushed onto the container mouth, the retaining ring can be pushed onto the press-fit cap. It encloses the latching elements from the outside in the form of a ring and thus fixes them in the locked position; it is then no longer possible for the latching elements to move outward and thus release the locking mechanism.

In a particularly preferred embodiment, which is considered to be independently inventive, an RFID chip is arranged on the press-fit cap, particularly preferably on its annular cover. This can be provided with a code or identifier that individually identifies the medication container and/or can contain further information regarding the contents, for example the medication or active ingredient composition, an eventual expiration date or the like. In particular, since such a chip can also be read without contact, automated handling of the medicament container and its contents, up to and including fully or partially automated medicament production in mixing systems or the like, can be achieved.

In an embodiment considered to be independently inventive, a closure system of the type mentioned above, preferably in conjunction with the embodiment explained above, is provided with a tamper-evident closure for the medicament container. The closure system may thereby be provided with an additional outer closure element in the manner of a disposable closure. This disposable closure, which can comprise, for example, a sealable lid that can be torn off or sealed, allows problem-free and reliable identification of whether or not the container has already been used for liquid transfer, and thus facilitates assignment of whether the container has already been “opened” and should therefore preferably be used for further liquid withdrawal until it is completely emptied and should therefore be disposed of.

In this embodiment, which is considered to be independently inventive, of the closure system for a medicament container, the interior of which is accessible via a container opening configured in the manner of a bottle mouth, this comprises:
a. a press-fit cap with an annular lid having a central opening, on the outer circumference of which a number of latching elements are arranged which can be brought into engagement with an outer bead arranged circumferentially on the container opening,
b. a one-piece closure stopper with a closure plate, on the first side of which a thickening is formed which completely fills the central opening of the annular cover and can be brought into engagement with the latter tend, and
c. a tamper-evident closure firmly attached to the outside of the press-fit cap with a tear-off ring to which a sealing plate is molded in its central region, completely covering the central opening of the annular cover.

The tamper-evident closure advantageously comprises a connecting segment extending between the tear-off ring and the sealing plate and integrally formed with the tear-off ring and/or the sealing plate via a number of predetermined breaking points, for fixed connection to the press-fit cap.

With regard to the medicament container, the interior of which is accessible via a container opening designed in the manner of a bottle mouth, the aforementioned object is achieved by providing it with a closure system of the type described above.

Also considered to be independently inventive is the use of a closure system of the type mentioned for a medicament container.

The advantages achieved with the invention consist in particular in the fact that the suitable design of the sealing stopper provides targeted sealing surfaces in the radial direction, i.e. towards the inner wall of the area of the bottle mouth surrounding the sealing stopper. These can have an additional sealing effect and thus improve the overall tightness of the system.

### BRIEF DESCRIPTION OF THE DRAWINGS

An embodiment of the invention is explained in more detail with reference to a drawing. Therein show:

FIG. 1 a medication container closed with a closure system,

FIG. 2 the medication container according to FIG. 1 in longitudinal section in exploded view,

FIG. 3 a longitudinal section of the medication container according to FIG. 1,

FIG. 4 a sealing stopper for the medication container according to FIG. 1,

FIG. 5 a top view of a press-fit cap of the closure system,

FIG. 6 a tamper-evident closure of the closure system in different views,

FIG. 7 a sequence of steps in attaching the closure system to the medication container of FIG. 1,

FIG. 8 the sealed medication container 1,

FIG. 9 the medication container 1 shown in FIG. 8 after the tamper-evident closure has been removed,

FIG. 10 the medication container 1 as shown in FIG. 8 in the connected state,

FIG. 11 a drug container closed with an alternative closure system,

FIG. 12 the sealing stopper of the alternative closure system according to FIG. 11,

FIG. 13 the press-fit cap of the alternative closure system according to FIG. 11,

FIG. 14 a pre-assembled system of press-fit cap and closure stopper with an alternative design of the closure stopper in longitudinal section,

FIG. 15 a longitudinal section of the medication container provided with the alternative closure system,

FIG. 16 an alternative embodiment of a tamper-evident closure for the medicament container according to FIG. 11, and

FIG. 17 a sequence of steps in attaching the alternative closure system to the medication container according to FIG. 11.

Identical parts are marked with the same reference signs in all figures.

### DETAILED DESCRIPTION OF THE DRAWINGS

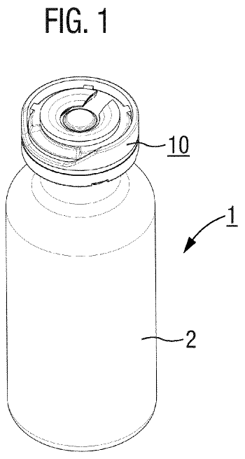

The drug container 1 as shown in FIG. 1, also referred to as a container or vial, is designed in the manner of a small bottle. It comprises an interior space 4 enclosed by a container wall 2, in which the medicament or the active ingredient is held. In the embodiment shown, the container wall 2 is made of glass, but can also be made of another suitable material, in particular one suitable for the safe storage of sensitive substances, such as a suitably selected plastic with or without a barrier layer. A “medical grade” plastic is particularly preferred, such as COP variants 690R®, 790R®, COC variants Topas® 8007S-04, 6013S-04, 6015S-04. The interior 4 is accessible via a container opening 6 in the form of a bottle mouth.

The medicament container 1 is especially designed for holding active substances or medicaments for which any material losses due to unauthorized release into the environment or surroundings are to be avoided as far as possible. This may be the case, for example, for substances that are toxic, hazardous to health or otherwise dangerous to the persons handling them, or also for particularly high-priced substances or active ingredients, such as those increasingly used in modern therapies. In order to keep such undesirable material losses to a minimum, the medication container 1 is equipped with a closure system 10 that closes the container opening 6 and is designed for particularly high tightness.

For its part, the closure system 10 has a multi-component design and comprises, as essential components, a closure stopper 12 for closing the container opening 6 and a fixing cap 14, with which the closure stopper 12 can be firmly attached to the container opening 6.

In the embodiment example, the container wall 2 of the medication container 1 is provided in the area of the container opening 6 with a circumferentially attached outer bead 16 as a fastening element. Adapted to this, the fixing cap 14 is designed as a press-fit cap 18, on the outer circumference of which a number of snap-on hooks or latching elements 20 are arranged to engage with the outer bead 16. When the press-fit cap 18 is fitted, it can thus be pushed or press-fitted onto the container opening 6, the latching elements 20 first being bent outwards by the outer bead 16 and then, after being pushed on further, engaging behind the outer bead and latching with it in the manner of a snap connection. Such a press-fit system is particularly easy to install and is therefore preferred; alternatively, however, a screw connection of the fixing cap 14 to the container wall 2 or another suitable connection could also be provided.

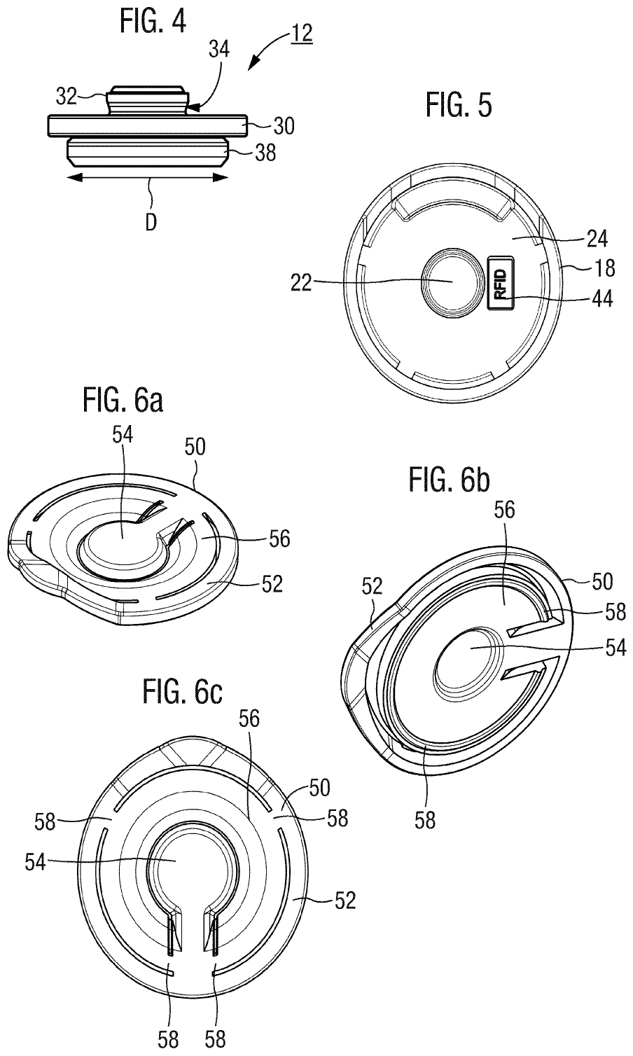

The press-fit cap 18 comprises an annular cover 24 having a central opening 22. The closure stopper 12, which is in itself of one-piece design, on the other hand comprises, in the manner of a basic element, a closure plate 30, on the first plate side of which there is integrally formed a thickened portion 32 which completely fills the central opening 22 of the annular cover 24 and can be brought into latching engagement therewith, as can be seen particularly clearly in the enlarged representation of the closure stopper 12 in lateral view in FIG. 4. The thickening 32 is provided in its connecting region with the closure plate 30 with a circumferential groove 34 forming an undercut. The closure stopper 12, which is in itself a single piece, is made of a material which is suitable and also comparatively soft and readily deformable, also with regard to the desired sealing purposes, in the embodiment shown of rubber or of TPE, preferably “medical grade”. This choice of material also ensures that the sealing stopper 12 can be pierced by means of a suitable instrument, for example a hollow needle, if required, i.e. if active substance is to be removed from the medicament container 1. Taking advantage of these material properties, in particular the deformability, the closure stopper 12 can be connected approximately firmly to the press-fit cap 18 by bringing the thickening 32 into the opening 22 in the annular cover 24 and the circumferential edge of the opening 22 subsequently engaging in the groove 34 and thus fixing the closure stopper fen 12 to the press-fit cap 18.

The sealing stopper 12 contributes to the particularly desirable sealing of the container opening 6 in two ways. On the one hand, a sealing effect is achieved, comparable with known systems, in that in the assembled system (as shown in the longitudinal section in FIG. 3) the closure plate 30, suitably adapted in its dimensions, in particular its outer diameter, to the mouth edge 36 of the container opening 6, is pressed onto the mouth edge 36 by the press-fit cap 18, which can be latched onto the mouth edge 36, with its edge of the closure stopper. This axial force effect, viewed in relation to the longitudinal axis of the container opening, means that the closure plate 30 can already develop a sealing effect as a result of the deformability of the material. In addition, however, the provision of radial force components, i.e. contact pressure forces, which press the closure stopper 12 in the radial direction against the inside of the container wall 2 in the region of its mouth, is also provided for a particularly increased sealing effect overall.

For this purpose, as is also clear from the enlarged illustration in FIG. 4, a radial sealing element 38 is formed on the second plate side of the closure plate 30. The radial sealing element 38 is adapted in its cross-sectional shape to the cross-sectional shape of the container opening 6 in the mouth area (in the embodiment example, both are round). In terms of its dimensions, it is also adapted to the clear width 1 of the container opening 6 and, with regard to the deformability of the material of the closure stopper 12, is designed to be slightly larger than the clear width 1 of the container opening 6. As a result, when the radial sealing element 38 is inserted into the container opening 6, a flat pressing or contact pressure is exerted on the inner wall of the container in the area of the container opening, taking into account the deformability of its material. With regard to common standards and usual norms for such components, the container opening can be suitably selected and dimensioned; for example, its clear width can be suitably selected to match the standard dimension “13 neck” (corresponding to an outer diameter of the container opening of 13 mm) or to match the standard dimension “20 neck” (corresponding to an outer diameter of the container opening of 20 mm).

In the embodiment shown, the press-fit cap 18 is made of a suitably selected plastic, namely polypropylene (PP), a polyolefin, cyclo-olefin copolymer (COC), cyclo-olefin polymer (COP) or polycarbonate.

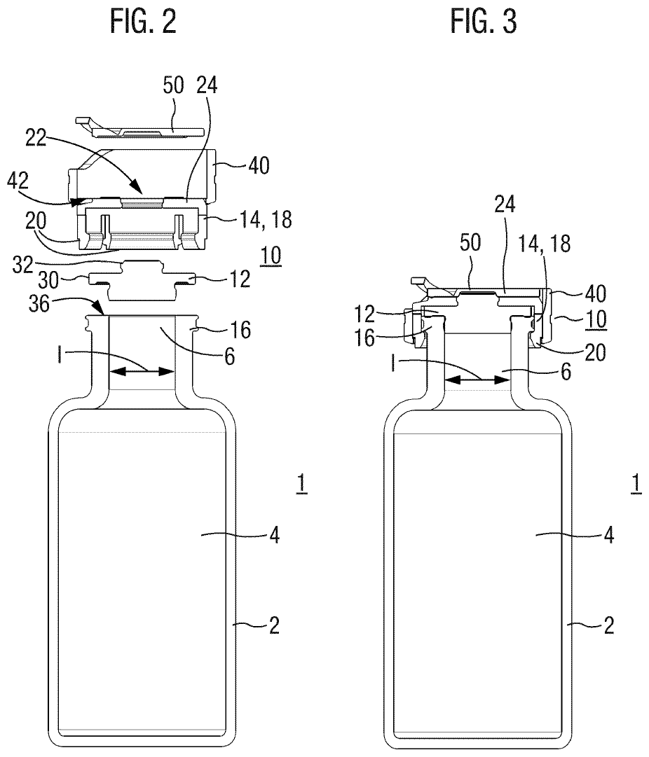

As a further component, as is again clear from the illustration in FIG. 2, the closure system 10 comprises a locking ring 40 which can be pushed onto the press-fit cap 18. This ring can be pushed onto the press-fit cap 18 from the outside, engaging around it, after the press-fit cap 18 has been press-fitted and has latched with the outer bead 16. This fixes the latching elements 20 radially so that they can no longer move outwards. This means that the latching of the press-fit cap 18 with the outer bead 16 can no longer be easily released and is thus fixed. On its side, the retaining ring 40 has a snap-on edge 42 formed on the inside and arranged circumferentially at the end, with which it can be fixed in a latching manner on the press-fit cap 18.

As can be readily seen from the top view of the press-fit cap 18 in FIG. 5, an RFID chip 44 is arranged on its annular cover 24. This can be provided with a coding or identifier which individually identifies the medication container and/or can contain further information relating to the contents, for example the medication or active ingredient composition, a possible expiry date or the like. In particular, since such a chip can also be read without contact, automated handling of the medicament container 1 and its contents, up to and including fully or partially automated medicament production in mixing systems or the like, can be achieved.

In a further embodiment considered to be independently inventive, the closure system 10, as can be readily seen in FIG. 2, for example, has a tamper-evident closure 50 as a component. This is intended to ensure, in the manner of a disposable closure, that the user can easily and reliably determine whether or not the medicament container 1 has already been used for liquid transfer, i.e. whether or not active substance has already been removed. It thus facilitates the assignment of whether the container has already been “opened” and should thus preferably be used for further liquid withdrawal until it is completely emptied and should thus be disposed of. The tamper-evident closure 50 is shown in detail in FIG. 6 in various views, namely in a side view from above (FIG. 6a), in a side view from below (FIG. 6b) and in a top view (FIG. 6c), therein firmly attached to the outside of the press-fit cap 18. In analogy to tamper-evident closures from the beverage sector, the tamper-evident closure 50 is provided with a tear-off ring 52, to which a sealing plate 54 is integrally formed in the central area of the tamper-evident closure 50. The sealing plate 54 is dimensioned and positioned in such a way that, in the assembled state, it completely covers the central opening 22 of the annular cover 24 and thus the exposed area of the closure stopper 12 accessible thereover. To access the inside of the medication container 1, i.e. to remove the active ingredient, the sealing plate 54 must first be removed so that the sealing stopper 12 can be pierced.

The tamper-evident closure 50 has a connecting segment 56 for fixed connection to the press-fit cap 18. This is arranged in segments around the sealing plate 54 between the tear-off ring 52 and the sealing plate 54 and is formed onto the tear-off ring 52 and the sealing plate 54 via a number of predetermined breaking points 58. This design ensures that initially the tamper-evident seal 50 can be firmly attached to the press-fit cap 18 as a whole. When the tear-off ring 52 is actuated, i.e. to remove the sealing plate 54, the predetermined breaking points 58 are then severed so that the ensemble of tear-off ring 52 and sealing plate 54 can be removed. Since the connecting segment 56 remains unchanged in its position on the press-fit cap 18, it is then easy to see that the seal has been broken and active substance has already been removed from the medication container 1.

A welding connection bead 58 is formed on the underside of the connection segment 56 for firmly connecting the tamper-evident closure 50 to the press-fit cap 18, as can be seen in the illustration in FIG. 6b. The welding connection bead 58 is provided for producing the connection with the upper side of the press-fit cap 18 by a welding process, for example by ultrasonic welding, and is designed accordingly. By placing it on the press-fit cap 18, pressing it on appropriately if necessary, and then subjecting it to an ultrasonic welding process, the connecting segment 56 and, with it, the tamper-evident closure 50 can then be connected overall to the press-fit cap 18 in a materially bonded manner.

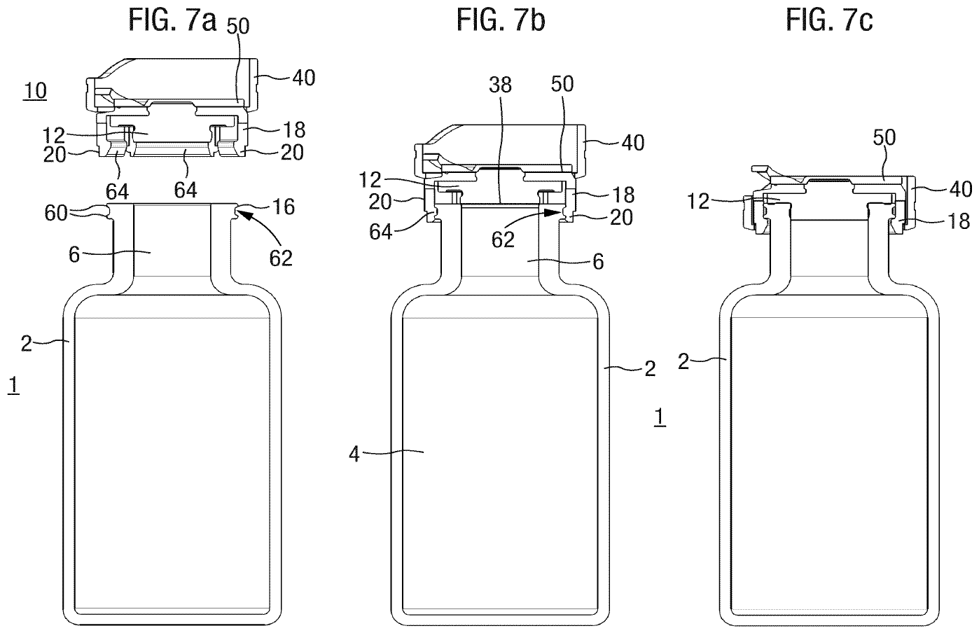

The attachment of the closure system 10 to the medication container 1 is shown in FIG. 7 using a sequence of steps. In a first step, shown in FIG. 7a, the closure system 10 is first preassembled in a sequence considered to be independently inventive. For this purpose, the tamper-evident closure 50 is firmly connected, in particular welded, to the press-fit cap 18, as described above, and the compression 32 of the closure stopper 12 is brought into snap-in connection with the opening 22 of the annular cover 24 of the press-fit cap 18, as also described above.

Starting from this pre-assembled state, the intermediate step shown in FIG. 7b can be carried out under certain circumstances and as required. In this intermediate step, the embodiment provided in the embodiment example is used, in which the outer bead 16 surrounding the container opening 6 on the outside in the mouth region is designed as a double bead. In this embodiment, the outer bead 16 comprises two individual beads 60 running parallel to one another, which are separated from one another by a groove 62 running between them. In such a design, it is possible to push the locking system 10 onto the container opening 6 in a first step only to such an extent that the latching hooks 64 formed on the respective latching elements 20 engage only in the groove 62 and initially lock the system there. In this intermediate state, which is shown in FIG. 7b, the sealing stopper 12 does not yet close the container opening 6, since the radial sealing element 38 has not yet penetrated into the interior of the container opening 6. Rather, in this state, a circumferential annular gap is formed between the radial sealing element 38 and the mouth of the container 1. Together with the circumferential gaps between the individual latching elements 20 of the press-fit cap 18, there is thus still a gas side connection between the interior 4 of the medicament container 1 and the external environment in this state.

This position can be used, for example, for freeze-drying, also referred to as lyophilization or sublimation drying, of the active ingredient in the medicament container 1. This is a now widespread process for the gentle drying of products, which is used for a large number of medicaments or active ingredients in order to preserve them. In such freeze-drying, it may be necessary to be able to release the gases or vapors produced, in particular water vapor, into the environment, and the positioning of the components shown in FIG. 7b offers such a possibility.

After this intermediate step, or possibly also directly after the pre-assembly shown in FIG. 7a if no such intermediate step is required, the closing process is then completed and the system is transferred to the fully closed state shown in FIG. 7c. For this purpose, the press-fit cap 18 is first moved further over the container opening, so that the sealing stopper 12 now penetrates with its radial sealing element 38 into the container opening 6 until the sealing plate 30 rests with its outer edge on the mouth of the container 1 and then, with slight deformation of the sealing plate 30 in the longitudinal direction of the container opening 6, the latching hooks 64 of the press-fit cap 18 engage below the second or lower individual bead 60. Subsequently, the locking ring 40 is then pushed downward so that it engages around the outside of the press-fit cap 18. This locks the latch elements 20 in position, and the medication container 1 in the position shown in FIG. 7c is securely closed.

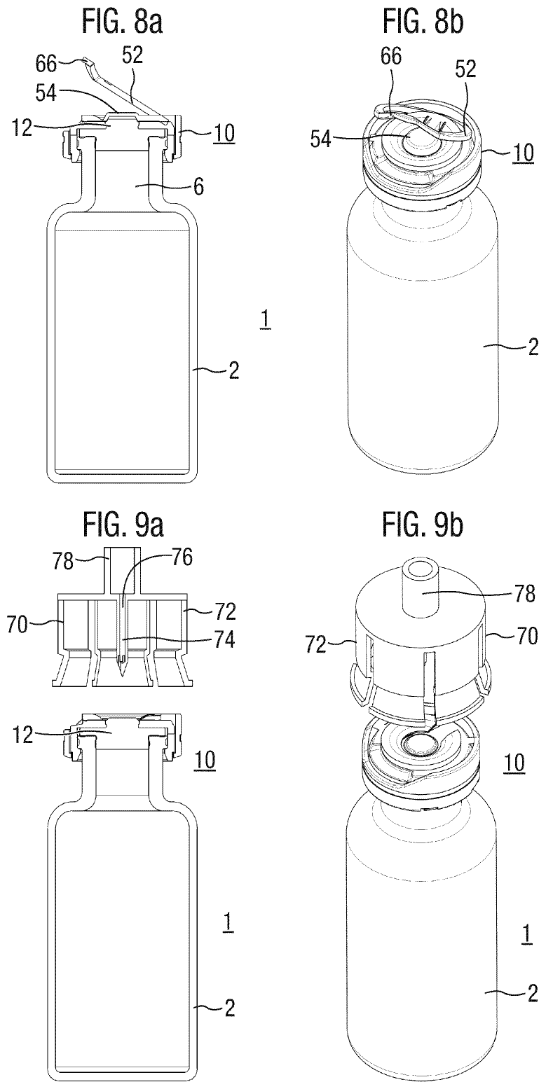

To open the medicament container 1 closed in this manner, as shown in FIG. 8a in longitudinal section and in FIG. 8b in perspective view, the tear-off ring 52 of the tamper-evident closure 50 is first lifted slightly by means of an integrated, slightly curved actuating segment 66, whereby the predetermined breaking points 58 connecting the tear-off ring 52 to the connecting segment 56 are severed. In this position, the tear-off ring 52 provides an engagement opportunity, for example for the user's finger, so that it can subsequently be easily removed completely, similar to the closure of a beverage can. In the process, the predetermined breaking points 58 connecting the sealing plate 54 to the connecting segment 56 are also severed, and the sealing plate 54 can be completely lifted off the press-fit cap 18 together with the tear-off ring 52. This exposes the part of the sealing stopper 12 located in the opening 22 and thus makes it accessible. At the same time, however, this also ensures that the opening is easily identifiable and the user can thus recognize that the active ingredient or the medication has already been accessed.

After the seal of the medication container 1 has thus been removed and the closure stopper 12 has been made ready for piercing, the “unlocked” medication container 1 can be connected to a suitable transfer or removal system. This is shown by way of example in FIG. 9a in longitudinal section and in FIG. 9b in perspective view. The removal element 70 shown there comprises a housing cap 72 suitably contoured and configured in its inner wall so that it can be snapped onto the closure system 10, in particular the retaining ring 40. Inside the housing cap 72, a hollow needle 74 is arranged as the actual removal element, by means of which the closure stopper 12 can be pierced. The inner channel 76 is connected on the media side to a connection piece 78 arranged on the outside of the housing cap 72, to which, for example, a hose or the like can be arranged.

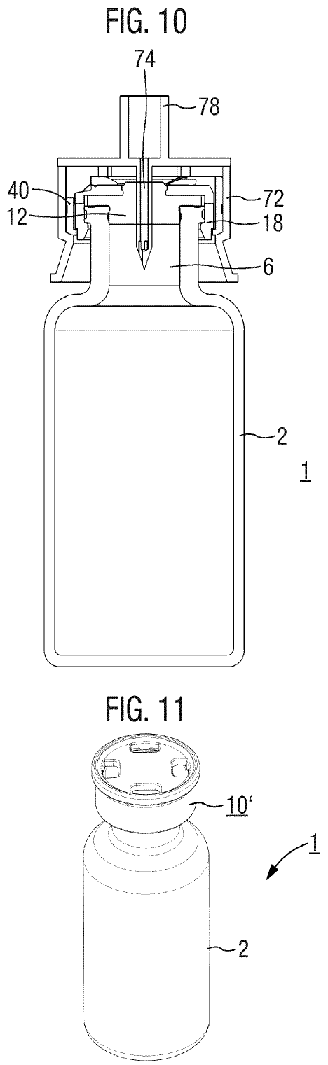

A medicament container 1 connected in this way to a transfer system or the like is shown with a pierced sealing stopper 12 in FIG. 10.

A medicament container 1 equipped with an alternative closure system 10′ is shown in FIG. 11 ff. The alternative closure system 10′, which is similar in basic concept to the closure system 10, differs from the former essentially in the precise design of the closure stopper 12′ and in the precise design of the tamper-evident closure 50′. All of these components are considered to be independently inventive and may be provided in any combination in accordance with the invention.

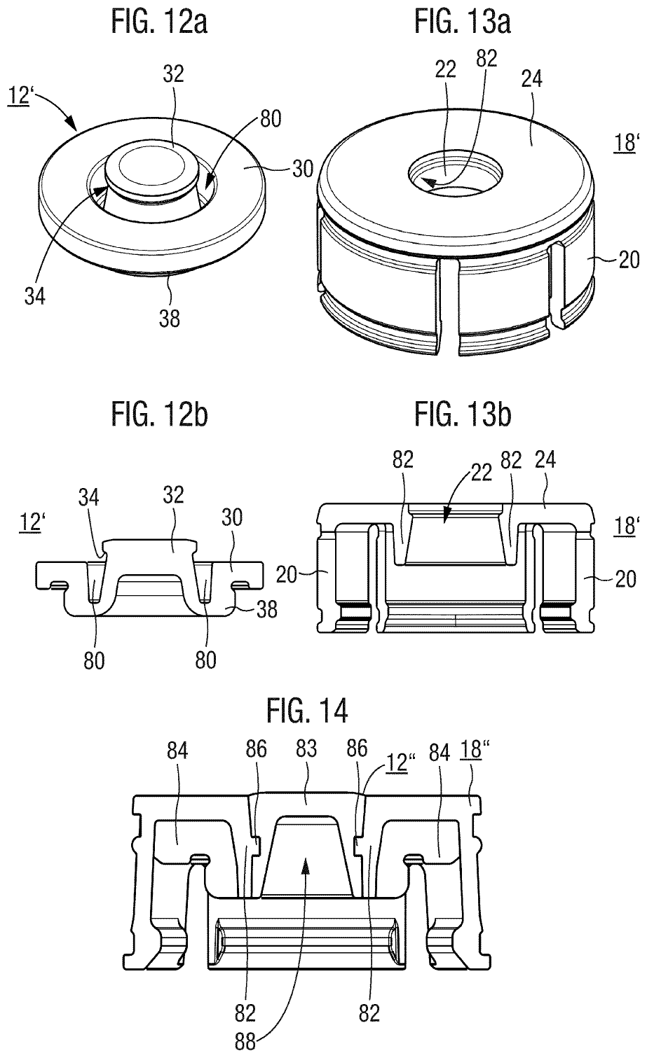

The closure stopper 12′ in the alternative embodiment of the closure system 10′ is shown in FIG. 12 in perspective view (FIG. 12a) and in longitudinal section (FIG. 12b). In this embodiment, too, it comprises, in the manner of a basic element, the closure plate 30, on the first plate side of which the thickening 32, which completely fills the central opening 22 of the annular cover 24 and can be brought into latching engagement therewith, is formed. As in the case of the closure system 10, in this embodiment the thickening 32 is also provided in its connecting region with the closure plate 30 with a circumferential groove 34 forming an undercut, and the closure stopper 12′, which is in itself a single piece, is made of a suitable material which is also comparatively soft and readily deformable, in the embodiment example of rubber or of TPE.

The sealing stopper 12′ is designed in an independently inventive embodiment for an even further improved sealing effect in the radial direction. For this purpose, the shape is selected in such a way that the central region of the sealing stopper 12′ forming the thickening 32 is surrounded by a groove-like or trench-like recess 80 extending deep into the sealing plate 30. Adapted to this, the press-fit cap 18′ in this embodiment, as shown in perspective view in FIG. 13a and in longitudinal section in FIG. 13b, has a reinforcing ring 82 formed on the ring cap 24 on its underside and surrounding the opening 22. When the two components are assembled, this reinforcing ring 82 is inserted into the recess 80 of the closure stopper 12′. The dimensions are thereby matched in such a way that the reinforcing ring 82 gives the radial sealing element 38 of the closure stopper 12′ increased strength and rigidity towards the outside, i.e. in the radial direction, and thus further improves the radial seal. In particular, due to the appropriately selected dimensions of the reinforcing ring 82, the radial sealing element 38 can be slightly deformed outwardly to a greater or lesser extent, thereby generating an additional contact force in the radial direction against the inner wall 4 of the medication container 1 in the region of the container opening 6.

A further variant of the ensemble of closure stopper 12″ and press-fit cap 18″, which is also regarded as independently inventive, is shown in longitudinal section in FIG. 14. The ensemble of these components is shown after they have been pre-assembled with one another, i.e. in the manner of an intermediate product prior to application to the medicament container 1 to be closed. In this variant, the sealing stopper 12″ shown hatched in FIG. 14 is designed in several parts or components, in the example shown in two parts. As the first component, it comprises a piercing element 83 arranged centrally with respect to the longitudinal axis, which is surrounded on the outside in the manner of a ring-like enclosure by the sealing element 84 provided as the second component. The reinforcing ring 82 of the modified press-fit cap 18″ is arranged annularly between these two in the pre-assembled state shown. Compared to the example described above, the reinforcing ring 82 and the recess 80 are extended towards the proximal side so that the recess 80 completely penetrates the thickness of the closure element 12″ and thus divides it into the two components 83, 84. In the pre-assembled state shown, the ensemble of closure element 12″ and press-fit cap 18″ thus has a three-component design (“3K variant”). In an embodiment regarded as independently inventive, the ensemble shown can also be produced from the outset in a 3K injection molding process.

The piercing element 83 is designed as a pierceable surface for, for example, a needle of a transfer system. Thus, by piercing this piercing element 83, access to the interior 4 of the container 1 can be created, via which active substance can be removed. Subsequently, the needle can be withdrawn again from the piercing membrane 83 thus formed, whereby the pierced surface closes again, preferably sealingly, due to the elastic properties of the stopper material. The desired sealing effect in the radial direction, on the other hand, is provided by the sealing element 84. Due to the functional separation of both components, in particular a functionally selected different design of both components, for example with regard to the choice of material and/or the specific material properties, can be provided with regard to the respective desired properties.

For stable connection of the press-fit cap 18″ to the piercing element 83 in the sense of reliable preassembly, a latching lip 86 is formed on the inside of the reinforcing ring 82, which engages in a corresponding groove on the outer shell of the piercing element 83.

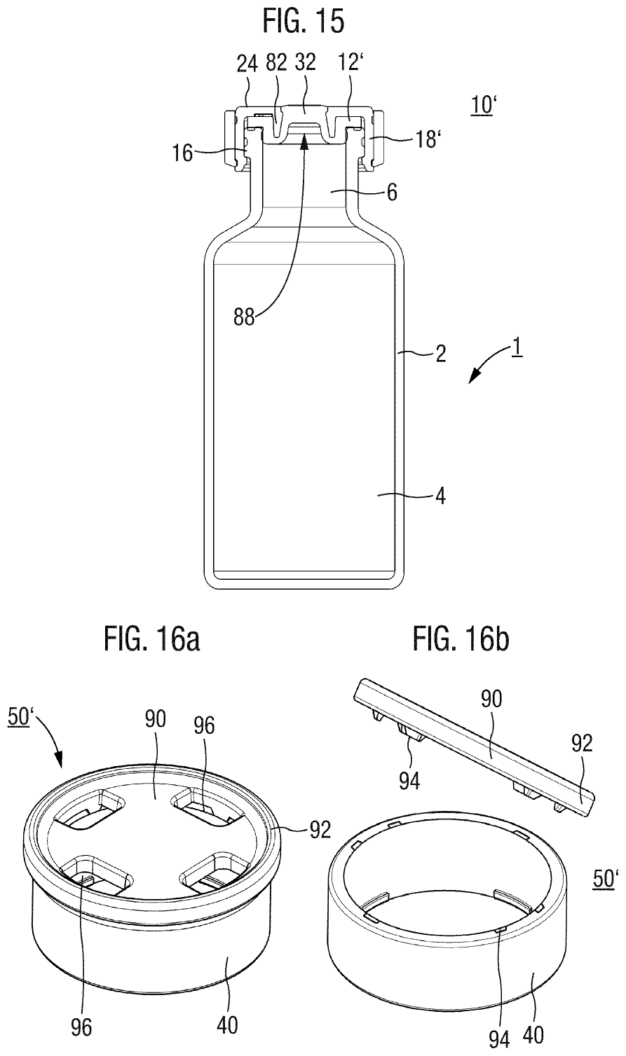

This mode of operation of the aforementioned components is also made clear once again in the illustration of the medication container 1 with the closure system 10′ in longitudinal section in FIG. 15. Here it can be clearly seen how the reinforcing ring 82 engages in the recess 80.

Furthermore, it can be seen from this illustration that the sealing stopper 12′ has a recess 88 directly below the thickening 32 in its central region on its side facing the interior 4 of the container 1. This means that the thickness of the sealing stopper 12′ in the area below the thickening 32 can be kept comparatively small without impairing the overall sealing effect, so that piercing with a hollow needle or similar is possible in a particularly simple manner.

Furthermore, in an embodiment also considered to be independently inventive, the tamper-evident closure 50′ is also alternatively designed in this embodiment, as shown in various views in FIG. 16. The tamper-evident closure 50′ comprises a cover plate 90 which is firmly connected to the retaining ring 40, which can be pushed onto the press-fit cap 18′, via a number of connecting webs 94 which are designed as predetermined breaking points, by means of a number of edges 92 which run around in the manner of a bead. The cover plate 90, which is provided with a number of venting openings 96 in its outer edge region, is designed to be two-dimensional in its central region, so that in the assembled state it completely covers and thus secures the underlying opening 22 of the press-fit cap 18′ and thus the thickening 32 of the sealing stopper 12′. To open the medicament container 1 closed in this way, the lid plate 90 is lifted by lifting the circumferential edge 92 at, whereby the connecting webs 94 designed as predetermined breaking points are torn off or severed. In this way, the part of the sealing stopper 12′ located in the opening 22 is also exposed and thus accessible in this system.

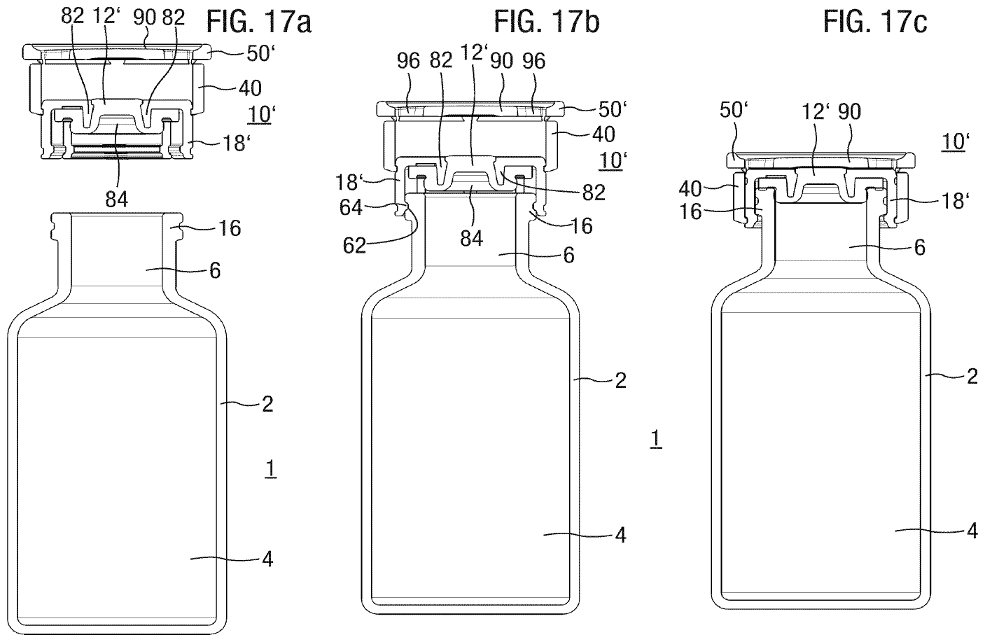

The attachment of the closure system 10′ to the medication container 1 is shown in FIG. 17 in analogy to the illustration in FIG. 7 using a sequence of steps. In a first step, shown in FIG. 17a, the closure system 10′ is also first preassembled here in a sequence considered to be independently inventive. For this purpose, the compression 32 of the closure stopper 12′ is first brought into snap-in connection with the opening 22 of the annular cover 24 of the press-fit cap 18′.

Starting from this pre-assembled state, the intermediate step shown in FIG. 17b can also be carried out here under certain circumstances and as required, for example to carry out freeze-drying or lyophilization. In this intermediate step, the embodiment provided in the embodiment example is used, in which the outer bead 17 surrounding the container opening 6 on the outside in the mouth region is designed as a double bead. In this embodiment, too, the closure system 10′ can, if necessary, be pushed onto the container opening 6 in a first step merely to such an extent that the latching hooks 64 formed on the latching elements 20 in each case only engage in the groove 62 and initially lock the system there. In this intermediate state, which is shown in FIG. 17b, the sealing stopper 12′ does not yet close the container opening 6, since the radial sealing element 38 has not yet penetrated into the interior of the container opening 6. Rather, in this state, a circumferential annular gap is formed between the radial sealing element 38 and the mouth of the container 1. Together with the circumferential gaps between the individual latching elements 20 of the press-fit cap 18′, there is thus still a gas side connection between the interior 4 of the medicament container 1 and the outer environment in this state.

After this intermediate step, or possibly also directly after the pre-assembly shown in FIG. 17a if no such intermediate step is required, the closing process is then completed and the system is transferred to the fully closed state shown in FIG. 17c. For this purpose, the press-fit cap 18′ is first moved further over the container opening, so that the sealing stopper 12′ now penetrates with its radial sealing element 38 into the container opening 6 until the sealing plate 30 rests with its outer edge on the mouth of the container 1 and then, with slight deformation of the sealing plate 30 as seen in the longitudinal direction of the container opening 6, the latching hooks 64 of the press-fit cap 18′ engage below the second or lower individual bead 60. Subsequently, the locking ring 40 is then displaced downward so that it engages around the outside of the press-fit cap 18′. This locks the latching elements 20 in position, and the medication container 1 in the position shown in FIG. 17c is securely closed.

### LIST OF REFERENCE SIGNS

1 Medication container
2 container wall
4 Interior
6 Container opening
10,10′ Closure system
12,12′ Sealing stopper
14 Fixing cap
16 Outer bead
18,18′ Press-fit cap
20 latch elements
22 Opening
24 annular cover
30 closure plate
32 thickening
34 groove
36 mouth edge
38 radial sealing element
40 security ring
42 snap rim
50,50′ Originality closure
52 Tear-off ring
54 sealing plate
56 connection segment
58 breaking point
60 single bead
62 groove
64 snap hooks
66 operating segment
70 removal element
72 housing cap
74 hollow needle
76 interior channel
78 connection piece
80 recess
82 reinforcement ring
83 piercing element
84 sealing element
86 lip
88 recess
90 lid plate
92 border
d Diameter
I light width

1. A closure system for a medicament container, whose interior is accessible via a container opening designed as a bottle mouth, the closure system comprising
a press-fit cap with a annular cover having a central opening, on an outer circumference of which ring a number of latch elements are arranged which can be brought into engagement with an outer bead fitted peripherally on the container opening, and
a closure stopper with a closure plate, on a first plate side of which there is integrally formed a thickened portion which completely fills the central opening of the annular cover and can be brought into latching engagement therewith, and on a second plate side of which there is integrally formed a radial sealing element having a cross section which is adapted to a clear width of the container opening and, with respect to the deformability of the material of the closure stopper, is chosen to be slightly larger than the clear width of the container opening.

2. The closure system of claim 1, the closure stopper of which is made in one piece.

3. The closure system of claim 1, wherein for the closure stopper a shaping is selected in such a way that the central region of the closure stopper forming the thickened portion is surrounded by a circumferential groove-like or trench-like recess extending into the closure plate.

4. The closure system of claim 3, the press-fit cap of which has a reinforcing ring formed on the annular cover on its underside, surrounding the opening and inserted into the recess of the closure stopper.

5. The closure system of claim 1, the press-fit cap of which is made of a plastic, preferably polypropylene (PP), a polyolefin, cyclo-olefin copolymer (COC), cyclo-olefin polymer (COP) or polycarbonate.

6. The closure system of claim 1, whose closure stopper is made of rubber or of TPE.

7. The closure system of claim 1, further comprising a locking ring which can be pushed onto the press-fit cap and can be fixed in a latching manner to the press-fit cap by means of a snap-on edge which is integrally formed on the inside and is arranged circumferentially at its end.

8. The closure system of claim 1, wherein an RFID chip is arranged on the annular cover of the press-fit cap.

9. The closure system of claim 1, for a medicament container, the interior of which is accessible via a container opening configured as a bottle mouth, wherein the closure system comprises:
a press-fit cap with an annular cover having a central opening, on the outer circumference of which annular cover a number of latch elements are arranged which can be brought into engagement with an outer bead fitted peripherally on the container opening,
a closure stopper with a closure plate, on the first plate side of which a thickened portion which completely fills the central opening of the annular cover and can be brought into latching engagement therewith, and
a tamper-evident closure which is fixed on the outside of the press-fit cap and has a tear-off ring on which in its central region a sealing plate completely covering the central opening of the annular cover is integrally formed.

10. The closure system of claim 9, wherein for the closure stopper the shaping is selected in such a way that the central region of the closure stopper forming the thickened portion is surrounded by a circumferential groove-like or trench-like recess extending into the closure plate.

11. The closure system of claim 10, the press-fit cap of which has a reinforcing ring formed on the annular cover on its underside, surrounding the opening and inserted into the recess of the closure stopper.

12. The closure system of claim 9, the tamper-evident closure of which comprises, for fixed connection to the press-fit cap, a connecting segment which extends between the tear-off ring and the sealing plate and is attached to the tear-off ring and/or to the sealing plate via a number of predetermined breaking points.

13. A medicament container, an interior of which is accessible via a container opening designed as a bottle mouth, which is provided with a closure system of claim 1.

14. A method comprising use of a closure system of claim 1 for a medicament container.
